(19) United States
(12) Patent Application Publication
(10) Pub. No.: US 2025/0128080 A1
(43) Pub. Date: Apr. 24, 2025

(54) THERAPEUTIC DEVICE

(71) Applicant: EAST LONDON ELECTRIC COMPANY LTD, London (GB)

(72) Inventors: Chris DAVIES, London (GB); Rob FIELD, London (GB); Jonathon TUCK, London (GB)

(21) Appl. No.: 18/685,119

(22) PCT Filed: Aug. 19, 2022
(86) PCT No.: PCT/GB2022/052154

§ 371 Date: Feb. 20, 2024

(30) Foreign Application Priority Data
Aug. 20, 2021 (GB) 2111950.8

(51) Int. Cl. A61N 2/00; A61N 2/02; G16H 40/63; G16H 40/67

(52) U.S. Cl. CPC A61N 2/008; A61N 2/02; G16H 40/63; G16H 40/67

(57) ABSTRACT

The present invention relates to a therapeutic device for the non-invasive treatment of human or animal bodies. Particularly, though not exclusively, the invention relates to a therapeutic device that selectively emits and maintains an energy field (electrostatic field, pulsed electromagnetic field or ultrasonic field) within a preselected frequency range for the effective alleviation of pain, inflammation and/or an alternative treatment. The device comprises an electronic circuit including a frequency generator for selectively producing electrical signals. The electronic circuit comprises a toroidal coil that is adapted to produce an oscillating electromagnetic field within a preselected frequency range in response to energisation by said electrical signals. The electronic circuit is constructed and arranged to adjust the electrical signals produced such that the coil selectively maintains the pulsed electromagnetic field within the preselected frequency range. The coil may be detachable. The detachable coil may allow removal, replacement or interchange of the coil with another coil type or a transducer to enhance the range of treatments and therapies offered by the device.

The present invention relates to a therapeutic device for the non-invasive treatment of human or animal bodies. Particularly, though not exclusively, the invention relates to a therapeutic device that selectively emits and maintains an energy field (electrostatic field, pulsed electromagnetic field and/or ultrasonic field) within a preselected frequency range for the effective alleviation of pain and/or inflammation, or for therapeutic effect.

Conventional treatments for symptoms of pain, inflammation, wound repair and other ailments typically involve pharmaceuticals, surgery, and/or applied therapies such as physiotherapy. These treatments are often administered or performed by trained medical staff or appropriately qualified individuals. They may be costly, invasive, cause side effects and increasingly such treatments are subject to delay leaving patients in pain while awaiting treatment. Pulsed electro-magnetic field therapy provides an alternative method of treatment as a non-invasive pain relief therapy. Pulsed electromagnetic field therapy involves the application of a magnetic field to damaged, inflamed or other targeted tissue. The premise of electromagnetic field therapy is to apply an induced magnetic field to a host, producing a spectrum of physiological benefits. The therapy causes interaction of the generated magnetic field with magnetic components of living tissue and results in ion flux through cell membranes. The utility of electromagnetic field therapy as a successful treatment for a variety of conditions is increasingly recognised and documented in a wide range of published international academic papers. However, some devices designed to apply electromagnetic field therapy lack effectiveness due to their construction and operating parameters. In order to achieve the desired results using existing devices, extensive and/or regular periods of treatment may be required to alleviate pain to the satisfaction of a patient. Additionally, the safety of some therapeutic devices providing pulsed electromagnetic fields for personal use is not guaranteed; in particular, those powered from a mains source.

Accordingly, it is an object of the present invention to provide a therapeutic device that emits a pulsed electromagnetic field for the alleviation of pain and/or inflammation that is both safe and easy to use as an effective therapeutic treatment.

According to a first aspect of the invention, there is provided a therapeutic device for the alleviation of pain, inflammation or for therapeutic effect, the device comprising:
an electronic circuit including a frequency generator for selectively producing electrical signals, said circuit comprising a toroidal coil that is adapted to produce an oscillating electromagnetic field within a preselected frequency range in response to energisation by said electrical signals; and
wherein the electronic circuit is constructed and arranged to adjust the electrical signals produced such that the coil selectively maintains the pulsed electromagnetic field within the preselected frequency range.

Preferably, the electronic circuit comprises a processor, wherein the processor may control the output of the frequency generator within the electronic circuit such that the coil selectively emits a pulsed electromagnetic field within the preselected frequency range.

Preferably, the electronic circuit comprises a feedback loop, wherein the feedback loop provides feedback to the processor regarding operational parameters of the coil such that the processor may selectively control the frequency generator to provide the required electrical signals and maintain the pulsed electromagnetic field within the preselected frequency range.

The feedback loop within the electronic circuit may further comprise detectors arranged to monitor parameters of the coil and feedback electrical signals to the processor. The electronic circuit or feedback loop may comprise measuring means arranged to measure parameters including but not limited to: resistance, current, temperature and the like. The processor may be programmed to adjust electrical signals produced by the circuit in response to one or more of the measured parameters.

Thus, the invention enables the therapeutic device to maintain the desired oscillating electromagnetic field by ensuring that the processor and the frequency generator provide the required electrical signals within the electronic circuit to enable the toroidal coil to produce the requisite pulsed magnetic field within the preselected frequency range. During use of the device, the toroidal coil is subject to resistive heating. The increased temperature can lead to an increase in resistance of the coil, which can in turn vary the electromagnetic output of the coil. The electronic circuit is constructed and arranged to adjust parameters of the electrical signals to compensate for this change in resistance so that the coil continues to emit the required pulsed magnetic field within the preselected frequency range. Advantageously, this enables the device to be used for extended time periods and/or with a succession of patients without the device overheating, or any detrimental effect on performance of the device.

Preferably, the therapeutic device is suitable for use on the human or animal body. In operation, the device produces a pulsed electromagnetic field that is directed through positioning or manipulation of the device over or alongside an area of the body under treatment, such that the magnetic field extends into the body to stimulate a localised area of tissue. The therapeutic device may be used curatively and/or preventatively. The device may be used to treat human or animal bodies to reduce or alleviate symptoms of pain and/or inflammation. Alternatively, or additionally, the therapeutic device may be used as a form of therapy or preventative treatment to assist sleep, manage anxiety, or combat certain adverse effects of aging.

The toroidal coil may be arranged within the device as an antenna. One end of the toroidal coil may be arranged within the electronic circuit to receive a selective input of electrical signals from the frequency generator. An opposing end of the toroidal coil may be connected to ground. Thus, the coil may act as an antenna when generating the pulsed magnetic field.

The electrical signal input to the coil from the frequency generator may be filtered by a filter located in the electronic circuit between the frequency generator and the coil. The electrical signal input to the coil from the frequency generator may be amplified by at least one amplifier located in the electronic circuit between the frequency generator and the coil.

The device may be arranged such that the coil may selectively emit a pulsed or modulated electromagnetic field having a frequency in the range 250 kHz to 350 KHz. The coil may selectively emit a pulsed or modulated electromagnetic field having a frequency in the range between around 270 KHz and 310 KHz in response to energisation by the electrical signals. The device may be arranged such that the coil may selectively emit an oscillating electromagnetic field having a frequency in the range 275 kHz to 305 KHz. The device may be arranged such that the coil may selectively emit an oscillating electromagnetic field having a frequency of around 290 KHz.

The therapeutic device may be used to alleviate pain and/or inflammation that is acute or chronic. The device may be used preventatively to substantially restrict injury or pain following exercise. The device may also be used for alternative therapies to aid wellbeing such as wound healing, blood treatment, sleep therapy or as a meditation aid. The therapeutic device may be used in the treatment of disease or anxiety. Each application or treatment may require a different treatment program. Different treatment programs may include a specific: optimum internal arrangement of electronic components including coil type (diameter, length of winding, number and type of windings, and the like); and/or applied oscillation frequency of the magnetic field; and/or specified treatment duration; and/or optimised treatment intervals.

The coil may be detachable. The detachable coil may allow removal, replacement or interchange of the coil with another coil type or a transducer. The coil may be coupled to the electronic circuit via electrical connectors. The device may comprise complementary electrical connectors. The electrical connectors of the device and coil/transducer can be complementary electrical connectors enabling removal and replacement of the coil/transducer or interchanging of different coils and transducers. The electrical connectors may comprise pins and sockets, and the coil/transducer may be plugged and unplugged from the device using the pins and sockets for secure attachment and selective removal of the coil from the electronic circuit within the device.

The device may comprise locating features facilitating the interconnection of the coil/transducer within the device. The locating features may further comprise locking features arranged to securely interconnect the coil/transducer within the electronic circuit.

The device may be operable with a plurality of different coils for generating a preselected unique magnetic field when energised. Each coil may generate a different electromagnetic field in response to energisation by the electrical signals. Optionally, the device parameters and coil type may be pre-selected according to the required therapy to be administered by the device. Advantageously, the type of coil and/or parameters of the generated pulsed magnetic field may be tailored to each specific application giving patients bespoke targeted treatment for their condition or requirements.

The processor may be programmable with a variety of different therapeutic programs. The processor may be programmable by manual or automatic means.

The processor may be electrically connected to a transmitter for the transfer of data to an external device. The transmitter may comprise wireless components including but not limited to Bluetooth, WiFi, Lorawan Sigfox or other Low Power Wide Area (LPWAN) service, ultrasound or light. The processor and transmitter may provide feedback transmittable to external monitoring services and applications. Such data fed from the device can be incorporated or used to support artificial intelligence (AI) systems or other services.

Thus, advantageously, the device may be configured for the transmission of treatment data to external devices, services or cognisant (AI) platforms to enable automatic revision of treatment pathways and/or predictive analytics for improvement of therapeutic outcomes.

The device may be used for different treatments which have predetermined characteristics enabling use of and treatment by the device to be customised in terms of duration and intensity. A therapeutic program may comprise treatment for variable periods of time using two or more selected frequencies, concurrently or serially, a variety of pulse types or modulations.

The device may comprise a control, which control may be operable to communicate with the processor and select the required program. A therapeutic program may comprise treatment using two or more selected frequencies. The control may comprise a directly wired connection within the device between the processor and a plurality of input controls. Alternatively, or additionally, the control may comprise an external control panel communicable via wireless means with the transmitter and the processor within the device. The control may enable selection of a variety of parameters including but not limited to: actuation commands, frequency selection, treatment programs, different frequencies concurrently or serially, treatment duration, intensity, pulse type and modulation, and the like.

The toroidal coil may be in the form of an annular ring. Dimensions and construction of the toroidal coil may be selected according to the specific application and electromagnetic field to be generated by the device.

The toroidal coil may be formed from a conductor wound around an annular core. The conductor may be a wire. The conductor may comprise a twisted pair of wires. Advantageously, the twisted wire pair arrangement increases the effect of the generated field when the coil is energised.

The toroidal coil may be formed from at least 10 metres of conductor wound in an annular formation. The toroidal coil may comprise around 15 metres of twisted pair wires.

The toroidal coil may comprise an inner diameter of at least 10 mm. The toroidal coil may comprise an inner diameter of between around 20 mm and 30 mm. The toroidal coil may comprise an outer diameter of at least 30 mm. The toroidal coil may comprise an outer diameter of between around 40 mm and 60 mm.

The device may be constructed and arranged to produce peak voltages in the between range 5 volts and 12 volts. The device may be constructed and arranged to produce peak voltages of around 12 volts.

The processor may be programmed to perform a calibration of the coil and/or the external environment. The processor may be programmed to conduct a frequency sweep of the coil in the environment of use, in incremental steps to calculate the optimum frequency at maximum power. The electronic circuit may be arranged to measure the current across a low value resistor to calibrate the frequency at which maximum current is measured. The frequency sweep may be provided at regular intervals of 5 Hz or less. The calibration frequency sweep may be provided at regular intervals of 1 Hz.

The therapeutic device may be a portable therapeutic device.

The therapeutic device may comprise a power source. The device may comprise an energy storage means. The device may comprise a port electrically connectable to the power source. The device may comprise a rechargable energy storage means. The therapeutic device may be powered by a direct current (DC) power source. The device may be powered by at least one cell. The device may be powered by at least one rechargeable cell. The device may comprise a converter for converting direct current (DC) to alternating current (AC).

Advantageously, creating a therapeutic device that is battery operable increases portability and ease of use since there is no requirement to take account of the location of the power supply wire during use of the device.

The therapeutic device may be a handheld therapeutic device.

The device may further comprise a housing to substantially enclose and protect the electrical components. Internal electrical components making up the therapeutic device may be sized and arranged to ensure that the outer housing of the device fits within an adult average hand size, such that the device may be manipulatable by a user. The longest dimension of the therapeutic device may be less than 15 cm. The longest dimension of the therapeutic device may be less than 12 cm. The longest dimension of the therapeutic device may be less than 10 cm. The longest dimension of the therapeutic device may be around 9 cm.

The therapeutic device may be a wearable therapeutic device.

The therapeutic device may comprise a removable attachment for attaching the device to a person or animal in use. An outer housing of the device may comprise attachment means for inter-engaging with a removable attachment such that a user may wear the device where appropriate for treatment. The removable attachment may include a strap.

A part of the outer housing of the device may be movable to create an opening allowing selective access to at least one of the battery and/or the coil. Therefore, the coil and/or battery may be selectively accessible and detachable for the replacement or interchange of the coil and/or the battery. The coil and/or the battery may be separated from other electronic components by internal separators within the housing to substantially restrict access to the internal electrical components of the device during selective replacement of the coil and/or the battery. The separators may comprise relatively thin moulded plastic walls containing access for a wired connection between the coil and/or the battery and the remainder of the electronic circuit.

The device may comprise a detachable extension member. The extension member may be selectively attachable to the therapeutic device to extend the reach of the device. For example, the extension member may facilitate self-administered therapy by an individual using the device on parts of the body that are difficult to reach, or for self-administered use by patients who have mobility constraints.

The therapeutic device may comprise communication means for communication with an external control. The processor may comprise a communication means for communication of information to/from the external control. The external control may be operable to communicate details of a treatment program and/or modify selected settings of the therapeutic device.

The therapeutic device may comprise wireless communication means for communication with an external control. The wireless communication means may comprise Bluetooth and/or Wi-Fi. The external control may comprise an application (or ‘app’) provided on digital media, such as a computer, laptop, tablet and/or smartphone. The ‘app’ may allow a user to enter details of the required therapy via a graphical user interface. The app may then analyse the data according to the software code and advise a plan or therapeutic treatment based on the data inputs.

Alternatively, the communication means may comprise a direct wired connection to the processor enabling the therapeutic device to be ‘plugged in’ to an external control.

The electronic circuit of the therapeutic device may comprise a circuit board containing electronic components. The electronic circuit board may comprise one or more electronic components selected from the group including but not limited to: temperature sensor, frequency generator, detector(s), amplifier(s), filter(s) and the like. The electronic components may be interconnected using electrical connectors in the form of wires and/or tracks.

The electronic circuit and/or circuit board may be spaced from the toroidal coil. The electronic circuit and/or circuit board and the toroidal coil may be mounted on opposing sides of a spacer. The spacer may comprise a lightweight plate formed from a non-electrically conductive material. Such an arrangement is advantageous since it spaces the electronic circuit from the toroidal coil to minimise interference therebetween. A further secondary benefit of this arrangement is that the coil is separated from the electrical components. Therefore, where the coil is accessible within and removable from the device, the electronic components are protected against accidental damage on the opposing side of the spacer.

The spacer may space at least a portion of the electronic circuit from the toroidal coil by a known distance. The spacer may be located between the toroidal coil and the circuit board. The spacer may comprise a mounting plate. Alternatively, the spacer may comprise a portion of housing or electronics enclosure. The circuit board and the toroidal coil may be spaced by a distance of at least 3 mm. The circuit board and the toroidal coil may be spaced by a distance of at least 5 mm.

The electronic circuit of the therapeutic device may comprise a temperature sensor. The processing unit may be pre-programmed with a threshold temperature and if the temperature sensor registers a temperature above the threshold, the processor may follow a safety program, such as automatic shutdown of the device to prevent damage from overheating.

The device may comprise at least one selectively attachable semiconductor transducer capable of emitting energy a preselected frequency with the appropriate power for a therapeutic treatment. Waveforms other than sinusoidal may be transmitted by the device using at least one transducer. The device may be configured to generate/transmit higher frequencies modulated by a lower frequency or combinations of lower frequencies. Modulation, waveforms types, and amplitudes may vary.

According to a second aspect of the invention, there is provided a therapeutic apparatus for the alleviation of pain, inflammation or for therapeutic effect, the apparatus comprising the device according to the first aspect of the invention and at least two coils selectively and interchangeably connectable to the device, wherein each coil is structured to generate different magnetic fields in response to electrical stimulation.

Optionally, the apparatus may comprise a plurality of coils interchangeably connectable with the device, wherein each coil is configured to generate a different magnetic field in response to electrical stimulation. Advantageously the apparatus of the invention can provide a single therapeutic device with multiple interchangeable coils offering different treatment options through the generation of varied magnetic fields enabling the optimum coil type and treatment cycle to be selected for a specific ailment or therapeutic effect.

Optionally the apparatus may comprise at least one transducer configured to transmit energy at a preselected frequency. The transducer(s) may be interchangeably connectable the device.

According to a third aspect of the invention, there is provided a therapeutic apparatus for the alleviation of pain, inflammation or for therapeutic effect, the apparatus comprising:
a device having an electronic circuit including a frequency generator for selectively producing electrical signals, said circuit comprising a connector for selective attachment of a coil;
a plurality of coils, wherein each coil has a complementary connector that is selectively connectable to the connector of the device and wherein each coil is configured to produce a unique oscillating electromagnetic field within a preselected frequency range in response to energisation by said electrical signals;
wherein the coils are interchangeable on the device via the complementary connectors such that the device is configurable and operable to produce a variety of preselected oscillating electromagnetic fields enabling the optimum treatment to be selected and generated for the alleviation of pain, inflammation or for therapeutic effect.

Each coil may be structured differently to produce a unique electromagnetic field. Each coil may be structured or configured differently to produce a unique electromagnetic field when energised by variation of at least one of the following parameters including but not limited to: shape of the core, dimensions of the core, diameter of the core (minor and/or major radius of the core), material of the core, numbers of windings (turns of wire per unit length), length of windings, type and arrangement of windings (e.g. twisted pair wires) and the like.

Each coil may be energised to produce an electromagnetic field that varies according to one or more of the following parameters including but not limited to: field shape, field strength, intensity, homogeneity, direction and the like.

Any feature of the device of the first aspect of the invention may be incorporated into the apparatus according to the third aspect of the invention.

The above-described first, second and third aspects of the invention may be combined with any other aspect, feature or embodiment described in the specification or shown in the figures where appropriate.

Embodiments of the invention are now described with reference to the following drawings in which:

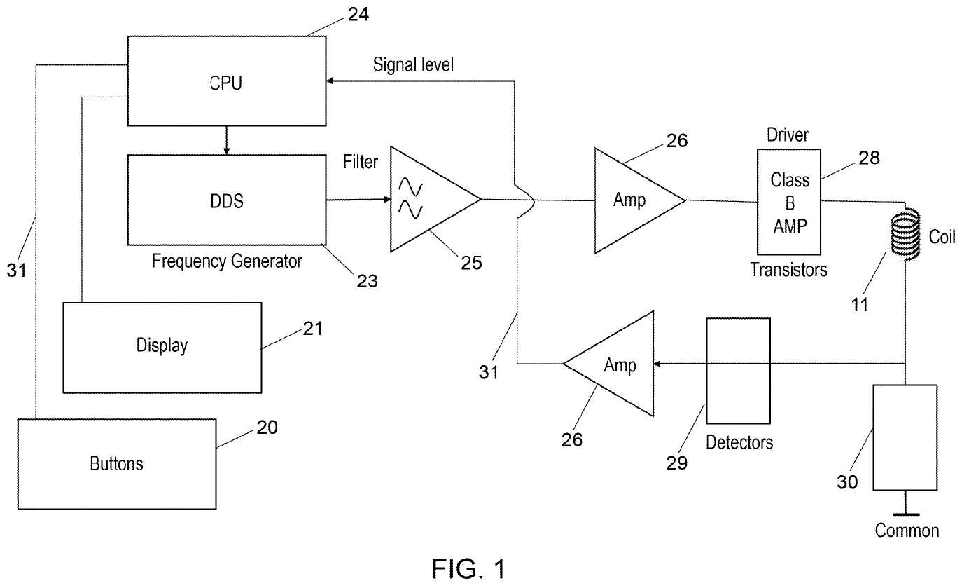

FIG. 1 is a circuit diagram showing the internal electronics of one embodiment of a therapeutic device according to the invention;

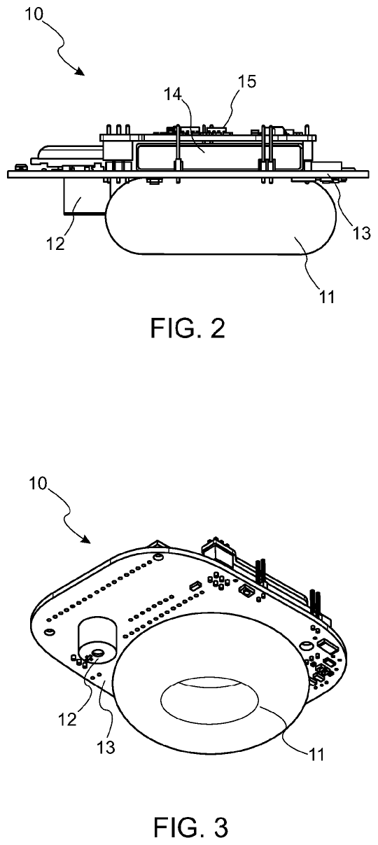

FIG. 2 is a side view of the internal components of the therapeutic device of FIG. 1;

FIG. 3 is a lower perspective view of the internal components of the therapeutic device of FIG. 2;

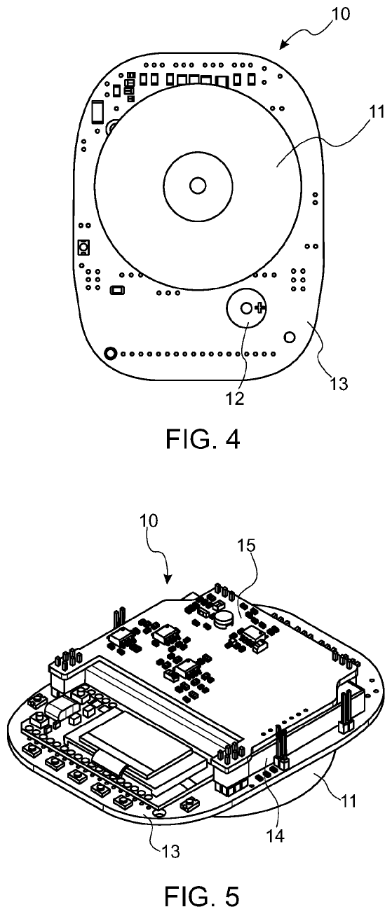

FIG. 4 is a plan view of the internal components of the therapeutic device of FIG. 2;

FIG. 5 is an upper perspective view of the internal components of the therapeutic device of FIG. 2;

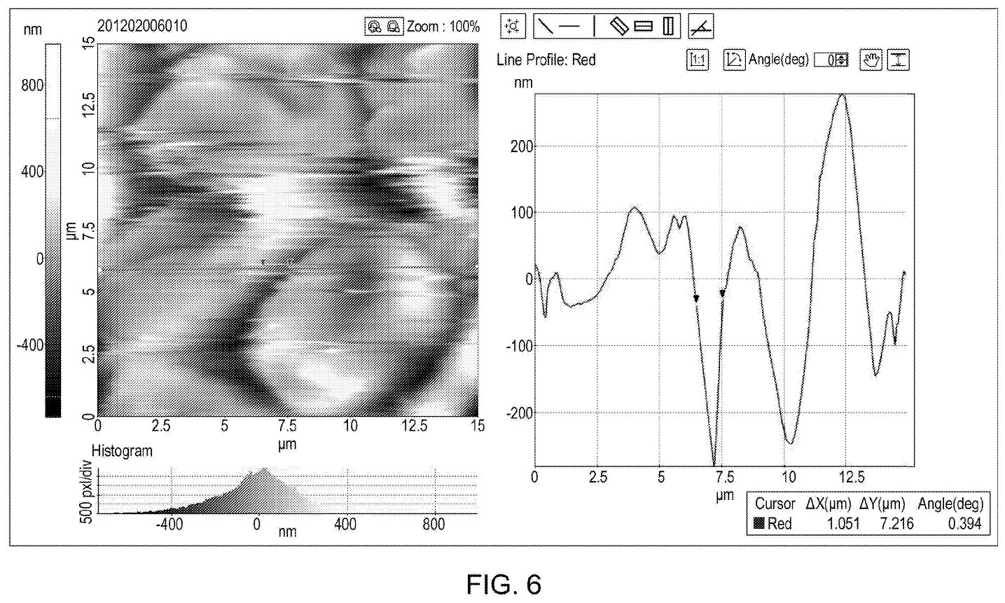

FIG. 6 is an image of red blood cells prior to treatment with the device captured by an atomic force microscope;

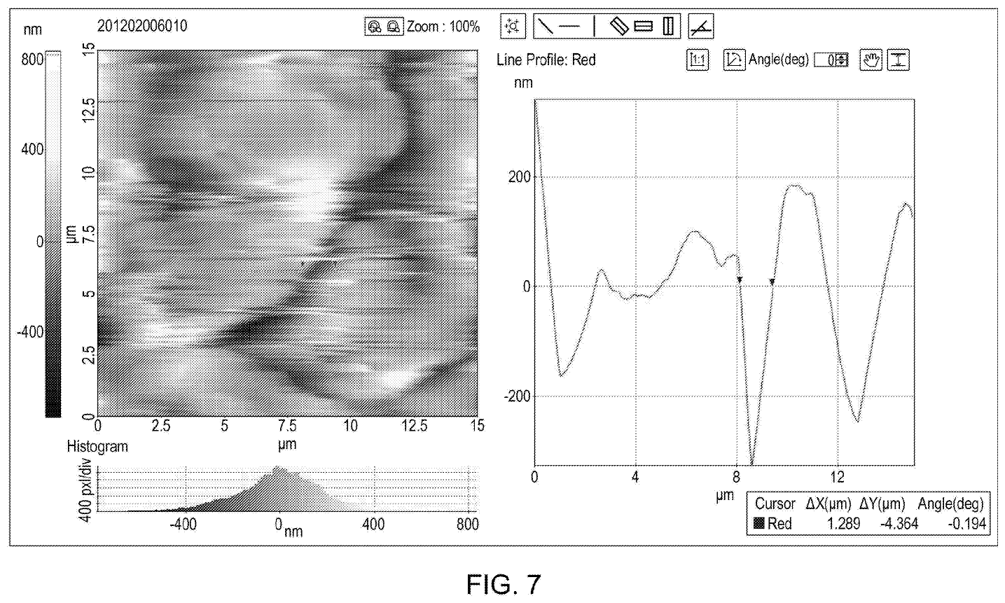

FIG. 7 is an image of red blood cells following 15 minutes of treatment with the device captured by an atomic force microscope;

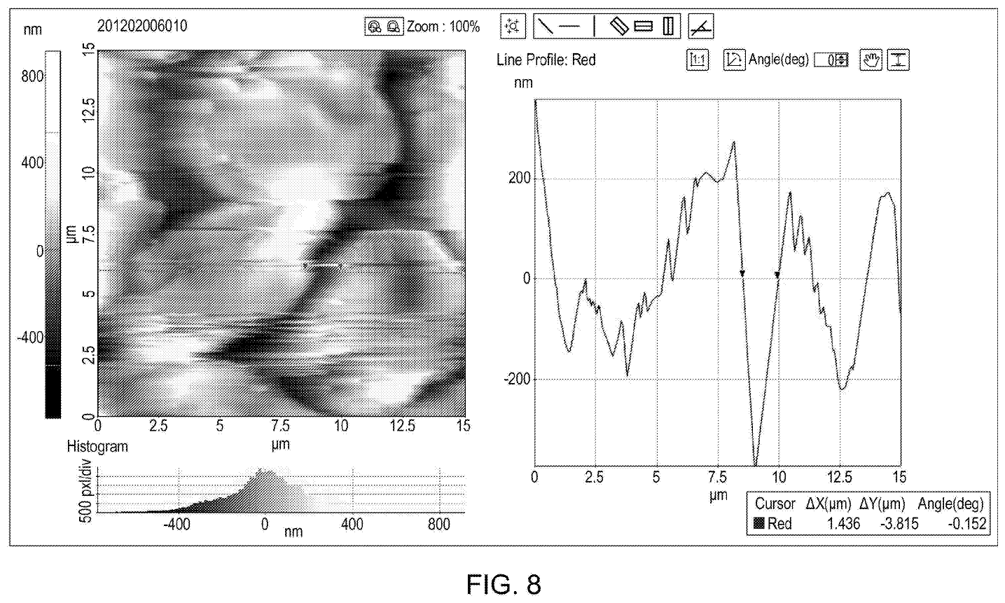

FIG. 8 is an image of red blood cells following two sets of 15 minute treatments with the device captured by an atomic force microscope;

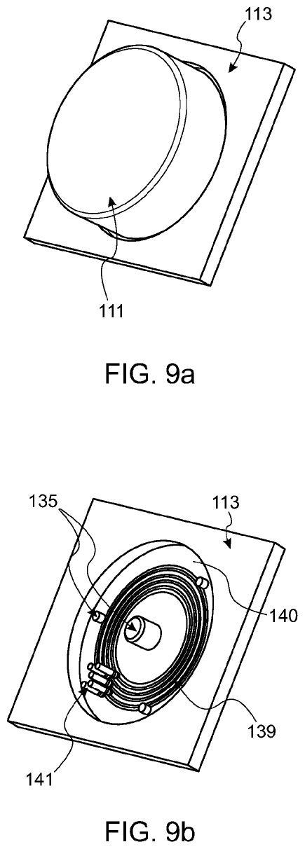

FIGS. 9a and 9b are partial perspective schematic views of another embodiment of the device carrying an interchangeable coil and with the coil detached from the device respectively; and

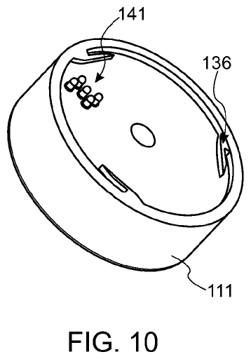

FIG. 10 is a partial perspective schematic view of a rear of a coil housing showing complementary connectors.

According to a first embodiment of the invention, the internal components of a therapeutic device are shown generally at 10 in FIGS. 2 to 5. The therapeutic device 10 is a handheld portable device provided with a moulded plastic interlocking outer housing to substantially enclose and protect the internal components and improve aesthetics. The outer housing has been omitted from FIGS. 2 to 5 for clarity to illustrate the internal arrangement of the components making up the therapeutic device 10.

The therapeutic device 10 comprises a spacer in the form of a centrally disposed plate 13 on which the internal components are mounted. One side of the centrally disposed plate 13 carries a toroidal coil 11 and a rechargeable battery 12 that are electrically connected to a circuit board 15 having electronic components mounted thereon (shown schematically in FIG. 1). The circuit board 15 is mounted on an opposing side of the centrally disposed plate 13.

According to one example of the coil 11 for the present embodiment, the toroidal coil 11 is made from 15 metres of twisted pair wire wound around an annular spool. The inner diameter of the coil 11 is 25 mm and the outer diameter is 50 mm. The circuit board 15 and electronic components are mounted adjacent to a spacer 14 that is provided to separate and space the toroidal coil 11 from the electronic components on the circuit board 15 by a distance of at least 5 mm to minimise interference therebetween. As evident from the FIGS. 2 to 5, the electronics and toroidal coil 11 are miniaturised, arranged in a space efficient manner that still enables effective performance of the coil 11 and robustly interconnected. As a result, the therapeutic device 10 is formed for use as a handheld device with dimensions (including the outer housing) of 9 cm long 6 cm wide and 5 cm deep.

As shown in FIG. 1, the electronic circuit board 15 carries a central processing unit (CPU) 24. The CPU 24 is electrically connected via wires 31 to a display 21 and controls in the form of buttons 20. The CPU 24 contains several programs using specific frequencies and durations. The required program is selectable by an operator or patient using the buttons 20. The display 21 provides information and data from the device 10, while the buttons 20 enable the device 10 to be controlled. The frequency is generated by a direct digital synthesiser (DDS) 23 locked to a quartz crystal for improved accuracy. The direct digital synthesizer 23 incorporates a quartz crystal frequency reference, an oscillator and a digital-to-analog converter. Output from the DDS 23 is fed through a filter to create a cleaner sine wave. The output from the filter 25 passes into an operational amplifier 26 that feeds into a class B amplifier 28 to step up the voltage from 5 volts to 12 volts. The output electrical signals then pass into the toroidal coil 11. There is a common ground 30 for the electronic components. The direct electronic connection of the toroidal coil 11 to ground 30 ensures that one end of the coil 11 has zero potential turning the coil 11 into antenna. Detectors 31 detect parameters of the output signal from the coil 11, which are fed into another amplifier 26 and back to the CPU 24 via electronic connectors in the form of wires or tracks 31 on the circuit board 15. Thus, a feedback loop is created, which can be used to vary the signal provided by the DDS 23 during use of the therapeutic device 10.

The outer housing (not shown) surrounds the internal components of the therapeutic device 10. The housing is smooth and has an ergonomic design such that it may be easily held and manipulated by hand. The outer housing includes an opening which accommodates the device display screen 12 for the presentation of selected information associated with the use of the device 10. The display screen 21 is electronically connected to the central processing unit 24 within the therapeutic device 10 and information displayed includes device 10 status (on/off), battery power status including charge warning light and program type. The housing is provided with an aperture or charging port, enabling insertion of a leading end of a power adapter for providing charge to the rechargeable battery 12 to provide power for the device 10. According to the present embodiment, the rechargeable battery carries at least 24 hours of power for the device 10 when maximally charged.

The device 10 is arranged to provide a selection of various treatment programs tailored to the needs of the patient. The various treatment programs differ in terms of the frequency of the magnetic field, duration of treatment and other criteria. Variables that may be considered to optimise selection of the most suitable program include: type of treatment (e.g. pain relief, anti-inflammatory, anti-aging, preventative, anti-anxiety, sleep aid etc) and individual characteristics of the patient (e.g. age, sex, location of treatment, physical condition, body mass index etc). Prior to use of the device 10 an operator or patient selects the recommended program.

An operator or patient requiring treatment using the therapeutic device 10 enters a suitable treatment program by selecting the appropriate buttons 20. The buttons also control actuation of the device 10. The buttons 20 enable initiation of the selected program via the CPU 24 which electronically communicates with the DDS 23. The device performs a calibration prior to commencement of the required program. The CPU 24 carries out a frequency sweep of the coil 11 and the environment in steps of 1 Hz to find the optimum frequency at maximum power. According to the present embodiment and described electronics arrangement, the optimum frequency is 290 500 Hz.

The DDS 23 and the filter 25 provide the required output frequency, which is amplified by the amplifiers 26, 28 to 12 volts to energise the toroidal coil 11. The coil 11 generates a magnetic field with the required frequency oscillation of 290.5 KHz. The magnetic field extends outwardly from the coil so that the device 10 may be ideally positioned proximate to the portion of the body to be treated.

The detectors 31 record parameters of the coil 11 and feedback to the CPU 24 via the amplifier 26. The CPU 24 processes data from the detectors 31 and provides the DDS 23 with an input adjusted as necessary to ensure that the frequency remains within the required preselected range for that particular treatment program. Thus, physical effects such as resistive heating of the coil 11 are negated by the feedback loop through the CPU 24 to maintain the desired magnetic field oscillating at the preselected frequency.

According to one embodiment a portion of the housing is removable to enable the coil 11 to be exchanged for another coil having different dimensions and length of winding such that the coil provides alternative properties and a different magnetic field. This allows the optimum coil to be selected for each treatment. Optionally the coil may be removed to allow for connection of a transducer configured to transmit energy at a preselected frequency.

According to an alternative embodiment, FIGS. 9a and 9b demonstrate schematically how a coil is mountable on an exterior of an external housing or enclosure 113. The internal electronics are provided within the housing 113, such that the external enclosure 113 acts as a spacer to separate the coil from the internal circuitry and reduce electrical interference therebetween. The housing 113 can contain electronics including a circuit board 15 and other electrical components described with reference to the previous embodiment. The coil is selectively attachable to an external portion of the housing 113 via complementary connectors 135, 136.

The coil is enclosed within a coil housing 111. The coil housing 111 is a substantially cylindrical shape having an underside with complementary connectors 136 in the form of angled retaining keyways and sprung electrical contacts 141 extending therefrom. As seen in FIG. 9b, an external part of the housing 113 is provided with a cylindrical recessed portion 140 that has a diameter slightly greater than a diameter of the coil housing 111. The recessed portion 140 is provided with a plurality of concentric circular conductive tracks 139 for power and data, which conductive tracks 139 are electrically coupled to the internal electronics within the housing 113. FIG. 9b also demonstrates schematically how the conductive tracks 139 engage with the sprung contacts 141 which are attached to the coil within the housing 111. The recessed portion 140 has complementary connectors 135 in the form of a centrally disposed protrusion and three radially spaced locator keys.

The housing 111 may also be used to accommodate a transducer configured to transmit energy at a preselected frequency. Thus the device 10 can allow the selective interconnection of transducer(s) to provide an alternative treatment or therapy.

In order to replace, interchange and engage a coil and/or connect a transducer, the underside of the coil housing 111 is aligned with the recessed portion 140. A centrally disposed hole is located over the centrally disposed protrusion 135 in the recessed portion 140. The coil housing 111 is twisted and pushed into the recessed portion 140 such that the three radially spaced locator keys 135 are urged into the complementary angled retaining keyways 136 of the coil housing 111 in order to securely attach and retain the coil housing 111 to the external housing 113 of the device.

A plurality of coils or transducers are provided within identical housings 111 such that different treatments may be administered via the same therapeutic device having a standard connection mechanism for various coils via the complementary connectors 135, 136. Each coil is located within the standard housing 111, but internally each coil is structured and configured to produce an electromagnetic field that varies according to one or more of the following parameters: field shape, field strength, intensity, homogeneity, direction and the like. The coil within each housing can be configured to produce the required field by variations in relation to one or more of the following parameters: shape of the core, dimensions of the core, diameter of the core (minor and/or major radius of the core), material of the core, numbers of windings (turns of wire per unit length), length of windings, type and arrangement of windings (e.g. twisted pair wires) and the like. Advantageously, according to the arrangement of components within this embodiment, the electronics are enclosed and protected within the device by the external housing 113, while access to the coil housing 111 is facilitated, enabling quick and easy interchanging of coil types as required for different treatments.

The therapeutic device of the invention 10 has been tested on ex vivo blood cells to observe and analyse biological effects of the magnetic field created by the device 10. An atomic force microscope (AFM) was used at the Rutherford Appleton Laboratories at the Harwell Campus, Oxford, to observe on a scale of nanometres the effects of the applied magnetic field generated by the device 10 on individual blood cells. FIG. 6 shows a photographic representation of the blood cells prior to applied magnetic field from the device 10. During treatment, the blood cells were observed to move apart and become spaced once the magnetic field was applied by the device 10. FIGS. 7 and 8 show an image of the blood cells following 15 minutes and 30 minutes respectively, of the applied magnetic field from the device 10. Inflammation is known to cause clumping and aggregation of cells. Therefore, the effects observed and captured by the AFM indicated cell reactions that could be responsible for reduction of inflammation and stimulation of cells to improve healing and reduce sensations of pain.

Exemplary data from use of the therapeutic device 10 when compared with other pain relief devices utilising magnetic field therapy, shows improvements in three main areas.

### 1) Reduced Recovery Time.

The therapeutic device 10 of the invention demonstrated reduction of pain after one day compared with up to ten days using alternative arrangements and parameters of magnetic coil.

### 2) Reduced Pain Threshold.

According to the McGill pain scale, the therapeutic device 10 of the invention is capable of a reduction of pain from an 8 on the numeric scale to 1-2. Alternative magnetic field treatments were not always effective and were not found to reduce pain by a similar amount.

### 3) Reduced Treatment Duration.

Improvements in the perception of pain were achievable in a much shorter timescale with the device 10 of the present invention. For example, the device 10 could be used once per day for between 15-30 minutes, compared with alternative treatments that require applied magnetic field therapy for 40 minutes to 1 hour, three times per day. Early patient responses and data indicates an improvement in physical condition when the therapeutic device 10 of the invention was compared with other available devices. Evidence collated shows an occurrence of a clinical response in relation to pain-relief, stress-relief, anti-oxidation (or anti-ageing) and quenched inflammation.

Research has demonstrated that all human cell types (including muscle, nerve, blood, lymphocytes and many others) and pathogens (viral, bacterial fungi and the like) are responsive to energy fields such as electromagnetic fields. The energy field applied by the device 10 will be varied according to the treatment offered. Other embodiments of the device 10 offer a variety of types of energy stimulation: magnetic, electromagnetic, electric, electrostatic, or sound energy fields might be applied alone or in combination. They are applied at appropriate frequencies, amplitudes and proximity to the patient. Where variable frequencies are used these might be linear, pulsed, modulated or combinations.

According to one case study, a mixed martial arts athlete reported suffering from an injury to his shoulder following exercise. His range of movement was limited to lifting his arms up to shoulder height beyond which the onset of pain limited further movement. The patient's shoulder was treated to 15 minutes of magnetic field therapy using the device at an oscillation of 290 kHz. The therapy was self-administered with the patient wearing a T-shirt. Following 15 minutes of therapy the patient was able to perform a press-up. Following another 15 minutes of therapy the patient had a full range of motion and use of the shoulder returned to normal. The following day the patient reported no pain or further problems.

Advantageously, the therapeutic device 10 works through fabric allowing the patient to undergo treatment while wearing clothes. Unlike some other conventional therapeutic devices on the market, the present invention does not require the arrangement of electrodes on a patient's body. Further, the use of a toroidal coil and arrangement of internal components within the therapeutic device 10 enable the dimensions to be minimised while still retaining the ability to effectively treat a patient.

Another advantage of the dimensions and weight of the device 10 is that the device 10 is wearable. According to some embodiments the device is provided with a strap and/or attachment means for securing the strap around/to the device 10 and the body of a human or animal.

Advantageously, the therapeutic device 10 is battery powered and therefore is easily manipulated and unencumbered by wires. Use of the device 10 is not limited by the length or position of a cable connector to mains power. This also improves safety of a patient during use of the device 10. Furthermore, the battery ensures that the device is portable and can be used by a person in any location without having to factor in the provision and orientation of an available power socket.

Thus, the hand-held device 10 is suitable for home use either by the patient or another individual. This in turn reduces the burden on health institutions and removes patients form a centralised care pathway involving the requirement to access physical treatment/recovery centres. Furthermore, this minimises the ongoing risk from COVID and other infections causing a rise in demand in beds and staff attention. The device 10 can enable health intuitions to minimise costs by shifting the responsibility of more minor ailments, aches and pains onto the patient/carer when appropriate to do so.

The device 10 may be used to provide an increased range of frequencies and wavelengths enabling a wider field of applications due to the interchangeable coil functionality extending the range from below 1 Hz to greater than 5 GHz. This increases applications for the device 10 by enabling hospitals to use the device 10 in conjunction with a range of drugs and nanoparticles to enhance their effects and minimize treatment time thereby further reducing costs.

Alternative embodiments include the display of additional information on the display screen 21 such as remaining program duration, magnetic field information (e.g. strength, frequency), external temperature, time, date and the like. According to one embodiment, an actuator is provided for selective operation of the device, wherein the actuator is provided on the display screen 21, which may be touch sensitive. According to other embodiments, the actuator comprises a separate manually operable switch. According to some embodiments, one or more disposable batteries may be used to provide power for the device. The housing may comprise a substantially continuous cover to enclose the internal electronics with a removable portion allowing access to disposable batteries for removal and replacement.

According to other embodiments, the device 10 is provided with a Bluetooth or Wi-Fi connection to enable wireless communication and remote commands to the central processing unit 24 within the device 10. One embodiment provides an application (or ‘app’) that contains details of the programs offered by the device 10. Thus, the devices are supportable by a related mobile phone or computer application which monitors the effectiveness of the treatment as well as controls and moderates patient usage.

A person intending to use the therapeutic device 10 enters their data into the ‘app’ via a graphical user interface (GUI) on a phone, tablet, PC or other device. The ‘app’ is pre-programmed to request or suggest the input of personal data including, but not limited to: site of injury, type and intensity of pain, preventative or curative treatment, personal statistics such as sex, height, weight and the like. Based on the inputted data, the ‘app’ selects a healing program or an injury prevention program that conforms with a user's individual requirements and calculates optimum magnetic field inputs and oscillation frequency alongside treatment duration and intervals.

According to other embodiments, the device 10 is adapted to communicate with an artificial intelligence (AI) system. Data collected from the device 10 and its associated application is usable for clinical research and “smart” adjustment of treatment through AI techniques. Use of AI techniques and predictive analytics in this way enable treatment programs to be automatically updated as a personalised medicine for each patient. All data is protected and stored in ways fully compliant with GDPR and other privacy and security regulations.

The computer program or ‘app’ can interact with the AI platform to offer predictive analytics to extend the treatment pathway for the patient based on datasets derived in related fields. The AI platform may also receive patient data from wearable devices such as a “smart” watch which can monitor real-time values for physical data. This physical data can include blood pressure, heart rate, blood oxygen saturation, exercise history and the like. Feedback of these values for AI and analytical purposes will also be used within the computer program or application to ensure the effectiveness of the device 10 and associated treatment. This also enables long term treatment plans to be improved without the immediate access to clinicians.

According to alternative embodiments, the housing or external enclosure is appropriately selected to allow for high volume manufacture and improved operational experience. For example, silicone mouldings provide improved feel and hygiene and can be sanitised without consequent material degradation.

The device 10 may be modified and configured to emit energy from a single source such as the coil 11 or a combination of sources such as the coil 11 and transducers. The device 10 may contain a biomarker sensor or another type of known sensor in order to diagnose and track cell health with microscopy photos or videos. The field emitted by the device 10 may be pulsed, modulated or stable. Alternative waveforms may be used, which clinical research demonstrates are most effective for a particular therapeutic treatment. The device 10 may be modified or used with other treatment options including electrical or ultrasound. These energies might be applied singly or in combination, pulsed or modulated for a variable period of time. The energy might be preselected or automatically derived (through local sensing, AI, feedback, or another technique) with a frequency range tested for effective treatment (alleviation of pain and/or inflammation, wound healing or for other therapeutic effect). The device 10 may be tailored for treatments within the input of clinicians specialising in genomic analysis. A full genome scan is currently cheaper than an MRI scan and can lead to effective detailed precision medicine. The portable devices 10 offer clinicians working in these areas an additional treatment option.

Scalar waves may be used in conjunction with the device 10 if appropriate for example in space or confined environments where mission supplies are extremely limited or depleted and/or where cells do not respond with positive outcomes to existing therapies, chemicals, or drugs and require a boost in efficacy at lower dosage to reduce payload. The device 10 may be used as an adjunct with nanoparticle infused drugs to localise and target specific areas of the body by placing an electric field at a specific frequency across the wound, infection or trauma site. Scalar information fields could have applications in editing DNA or genomic studies editing nucleotides in mice for a cost effective way to develop a clinical trial colony. The transmission of the energy field or fields concerned will be through the most appropriate method for the treatment concerned. This might be but not exclusively a wire coil, a transducer, direct electrical conductor, vibrating electromagnetic surface, electrically heated element or light emitting device. It could also be a plasma ball generator and emitter or long wire woven into a space suit for use on the mission to Mars or the Moon.

Modifications and improvements can be made without departing from the scope of the invention. Relative terms such as are used for illustrative purposes only and are not intended to limit the scope of the invention. Exemplary data and numerical examples used in the detailed description of the invention are illustrative only and are not intended to be limiting.

The effects of energy stimulation on cells vary considerably and other health benefits may be covered by this application. The detailed description of the invention indicates an example of a device 10 which has been proven to offer pain relief. This does not restrict the scope of this application exclusively in terms of size, construction, field type, field power, signal or waveform type, energy focus or any other parameter which research or clinical trials are shown to effect the outcome. Other health benefits than pain relief are also covered. There is evidence of a reduction of inflammation, prevention of illness, sleep function regulation for sleep induction, and infection control.

1. A therapeutic device for the alleviation of pain, inflammation or for therapeutic effect, the device comprising:
an electronic circuit including a frequency generator for selectively producing electrical signals, said circuit comprising a toroidal coil that is adapted to produce an oscillating electromagnetic field within a preselected frequency range in response to energisation by said electrical signals; and
wherein the electronic circuit is constructed and arranged to adjust the electrical signals produced such that the coil selectively maintains the pulsed electromagnetic field within the preselected frequency range.

2. A therapeutic device as claimed in claim 1, wherein the electronic circuit comprises a processor, and wherein the processor controls the output of the frequency generator within the electronic circuit such that the coil selectively emits a pulsed electromagnetic field within the preselected frequency range, and wherein the electronic circuit comprises a feedback loop, and wherein the feedback loop provides feedback to the processor regarding operational parameters of the coil such that the processor may selectively control the frequency generator to provide the required electrical signals to maintain the oscillating electromagnetic field within the preselected frequency range.

3. A therapeutic device as claimed in claim 2, wherein the feedback loop within the electronic circuit may further comprise detectors arranged to monitor parameters of the coil and feedback electrical signals to the processor, wherein the processor adjusts electrical signals produced by the circuit in response to the measured parameters.

4. A therapeutic device as claimed in any preceding claim, wherein one end of the toroidal coil receives an input of electrical signals from the frequency generator and an opposing end of the toroidal coil is connected to ground such that the toroidal coil is arranged within the device as an antenna.

5. A therapeutic device as claimed in any preceding claim, wherein the coil is adapted to emit a pulsed electromagnetic field having a frequency in the range between around 250 KHz to 350 KHz in response to energisation by the electrical signals.

6. A therapeutic device as claimed in any preceding claim, wherein the processor is programmable to provide different therapeutic treatments having varying duration and intensity and enabling selection of the suitable therapeutic treatment by a user.

7. A therapeutic device as claimed in any preceding claim, wherein the coil is detachable from the electronic circuit enabling removal and replacement of the coil, and thereby facilitating selection of the optimum coil according to the specific treatment using the therapeutic device.

8. A therapeutic device as claimed in any preceding claim, wherein the toroidal coil is formed from at least 10 metres of a pair of twisted wires wound around an annular insulator.

9. A therapeutic device as claimed in any preceding claim, wherein the toroidal coil comprises an inner diameter of between around 20 mm and 30 mm and an outer diameter of between around 40 mm and 60 mm.

10. A therapeutic device as claimed in any preceding claim, wherein the processor is programmed to perform a calibration of the coil and/or the external environment by conducting a frequency sweep of the coil in the environment of use, in incremental steps to calculate the optimum frequency at maximum power.

11. A therapeutic device as claimed in any preceding claim, wherein the device is portable and the dimensions of the device are minimised such that the device can fit within a hand of an average adult.

12. A therapeutic device as claimed in any preceding claim, wherein the device is portable and the longest dimension of the device is less than 15 cm.

13. A therapeutic device as claimed in any preceding claim, wherein the device is a wearable therapeutic device comprising a removable attachment for attaching the device to a person or animal in use.

14. A therapeutic device as claimed in any preceding claim, further comprising a selectively attachable extension member to extend the reach of the device and facilitate self-administered therapy.

15. A therapeutic device as claimed in any preceding claim, the device further comprising wireless communication means for communication with an external control to enable the external control to provide remote commands to the device via the wireless communication means.

16. A therapeutic device as claimed in any preceding claim, wherein components of the electronic circuit are spaced by at least 4 mm from the toroidal coil to minimise interference therebetween.

17. A therapeutic device as claimed in claim 16, comprising a spacer located between components of the electronic circuit and the toroidal coil, wherein the spacer comprises a lightweight electrically non-conductive material to substantially restrict interference between the coil and other electronic components of the device.

18. A therapeutic apparatus for the alleviation of pain, inflammation or for therapeutic effect, the apparatus comprising the device according to any one of claims 1 to 17 and at least two coils selectively and interchangeably connectable to the device, wherein each coil is structured to generate different magnetic fields in response to electrical stimulation.

19. A therapeutic apparatus as claimed in claim 18, wherein the apparatus further comprises at least one transducer configured to transmit energy at a preselected frequency and wherein the transducer(s) is interchangeably connectable the device according to any one of claims 1 to 17.

20. A therapeutic apparatus for the alleviation of pain, inflammation or for therapeutic effect, the apparatus comprising:
a device having an electronic circuit including a frequency generator for selectively producing electrical signals, said circuit comprising a connector for selective attachment of a coil;
a plurality of coils, wherein each coil has a complementary connector that is selectively connectable to the connector of the device and wherein each coil is configured to produce a unique oscillating electromagnetic field within a preselected frequency range in response to energisation by said electrical signals;
wherein the coils are interchangeable on the device via the complementary connectors such that the device is configurable and operable to produce a variety of preselected oscillating electromagnetic fields enabling the optimum treatment to be selected and generated for the alleviation of pain, inflammation or for therapeutic effect.

21. A therapeutic apparatus according to any one of claims 18 to 20, wherein each coil comprises a conductor wound around an annular core and wherein each coil is structured or configured differently to produce a unique electromagnetic field when energised, by variation of at least one of the following parameters including but not limited to: shape of the core, dimensions of the core, diameter of the core (minor and/or major radius of the core), material of the core, numbers of windings, length of windings, type and arrangement of windings.

22. A therapeutic apparatus according to any one of claims 18 to 21, wherein each coil is selectively energised to produce a unique electromagnetic field that varies according to one or more of the following parameters including but not limited to: field shape, field strength, intensity, homogeneity and direction.
